(19) United States
(12) Patent Application Publication
(10) Pub. No.: US 2024/0110241 A1
(43) Pub. Date: Apr. 4, 2024

(54) A Method Of Characterising An Immunosuppressive Neutrophil

(71) Applicant: AGENCY FOR SCIENCE, TECHNOLOGY AND RESEARCH, Singapore (SG)

(72) Inventors: Lai Guan Ng, Singapore (SG); Shu Feng Melissa Ng, Singapore (SG); De Li Leonard Tan, Singapore (SG); Florent Benoit Claude Ginhoux, Singapore (SG)

(21) Appl. No.: 18/256,303

(22) PCT Filed: Dec. 7, 2021
(86) PCT No.: PCT/SG2021/050767

§ 371 Date: Jun. 7, 2023

(30) Foreign Application Priority Data
Dec. 15, 2020 (SG) 10202012586X

(51) Int. Cl. C12Q 1/6881

(52) U.S. Cl. CPC C12Q 1/6881; C12Q 2600/158; G01N 2800/52; G01N 2800/56

(57) ABSTRACT

The invention relates to a method of detecting and/or characterising an immunosuppressive neutrophil comprising determining and/or measuring the expression of one or more anti-apoptotic markers in a neutrophil population. In an embodiment, the immunosuppressive neutrophil is a polymorphonuclear myeloid-derived suppressor cell (PMNMDSC) and the anti-apoptotic marker is TNF-related apoptosis-inducing ligand (TRAIL)-receptor 3 (TRAILR3/TNFRSF10C) and/or decoy TNF-related apoptosis-inducing ligand (TRAIL)-receptor (dcTRAILR1). Also disclosed are methods of evaluating the progression of a proliferative disease in a subject, methods of evaluating the efficacy of a treatment regimen for a proliferative disease in a subject, methods of treating a proliferative disease in a subject, and kits thereof.

### TECHNICAL FIELD

The present disclosure relates broadly to methods of detecting, sorting, and/or characterising immunosuppressive immune cells. In particular, the present disclosure relates to methods of detecting, sorting, and/or characterising immunosuppressive neutrophils.

### BACKGROUND

Neutrophils have been characterized to have anti-tumorigenic properties, with several preclinical cancer mouse models showing that they can hinder tumour growth through their production of cytotoxic reactive oxygen species. However, there is growing recognition that neutrophils play important roles in tumour growth, progression and metastasis in human cancers. Circulating neutrophil-to-leukocyte ratios (NLRs) have been utilized to predict patient outcomes, with increased NLR correlating strongly with poor response to treatment and prognosis. Within the tumour microenvironment, increased percentages of tumour infiltrating neutrophils are found to strongly correlate with poor overall survival (OS) and recurrence-free survival (RFS) across multiple solid cancer types. Additionally, tumour infiltrating neutrophils gain tumour-promoting abilities, such as the upregulation of immunosuppressive molecules and cell surface markers that promote immune evasion of the cancer cells. Thus, they are typically classified as the immunosuppressive PMN-MDSC population. Given that they make up a significant proportion of immunosuppressive cells within the tumour microenvironment (TME), depletion of tumour PMN-MDSC has been an attractive target as a single therapeutic, or in combination with checkpoint inhibitors to potentiate effective anti-tumour immunity.

However, cell surface markers utilized to denote PMN-MDSCs are the same markers used to identify normal circulating neutrophils both in mouse and human. As such, the ability to properly characterize and subsequently target PMN-MDSC in the cancer state in both pre-clinical mouse models, as well as in human patients, have been hampered due to the lack of specific surface markers to distinguish the PMN-MDSC from normal neutrophils. Additionally, two other factors add to the importance of specifically targeting tumour-infiltrating PMN-MDSCs. First, studies have shown that normal circulating neutrophils that are not reprogrammed into PMN-MDSCs within the tumour might retain anti-tumour function, making full depletion of all neutrophils disadvantageous even though it will remove tumour-infiltrating PMN-MDSC. Second, complete depletion of all neutrophils will dampen the ability of the immune system to fight off secondary infections. As such, current therapeutics with myeloablative abilities will deplete tumour PMN-MDSCs, but will also target normal neutrophils, leading to unwanted morbidity and mortality in cancer patients.

Taken together, there is an immediate need to identify specific markers of PMN-MDSCs in mice and human that can be used to study their function as well as targeted as drug candidates to selectively deplete this population to improve patient outcomes. That is, there is a need to provide a method of detecting and/or sorting and/or characterising an immunosuppressive immune cell, such as immunosuppressive neutrophil.

### SUMMARY

In one aspect, there is provided a method of detecting and/or characterising an immunosuppressive neutrophil comprising determining and/or measuring the expression of one or more anti-apoptotic marker in a neutrophil population.

In some embodiments, the immunosuppressive neutrophil is a polymorphonuclear myeloid-derived suppressor cell (PMN-MDSC).

In some embodiments, the anti-apoptotic marker is TNF-related apoptosis-inducing ligand (TRAIL)-receptor 3 (TRAIL-R3/TNFRSF10C; in human) and/or decoy TNF-related apoptosis-inducing ligand (TRAIL)-receptor (dcTRAILR1; in mouse).

In some embodiments, the method further comprises determining and/or measuring the expression of one or more markers related and/or capable of inducing immunosuppression of an immune cell.

In some embodiments, the markers related and/or capable of inducing immunosuppression of effector immune cells comprises one or more of the following markers: CD274 (PD-L1), VSIR (VISTA), LILRB4, ARG1, PTGS2 (COX2), or NOS2.

In some embodiments, the method further comprises determining and/or measuring the expression of one or more markers that negatively regulate inflammatory function, optionally negatively regulate innate inflammatory function.

In some embodiments, the markers that negatively regulate innate inflammatory function comprises one or more of the following markers: Dcir2, Pir-a/b, or Clec12a.

In some embodiments, the method further comprises determining and/or measuring the expression of one or more markers of metabolic ectoenzymes that create immunosuppressive tumour microenvironment (TME).

In some embodiments, the \markers of metabolic ectoenzymes that create the immunosuppressive TME comprises one or more of the following markers: CD39, or CD73.

In some embodiments, the method further comprises determining and/or measuring the expression of markers comprising one or more of the following markers:
(in mouse) CD11b⁺, Gr-1^{hi}, Ly6C^{low}, Ly6G⁺, CD101^{+/-}, optionally wherein CD101⁺ refers to mature neutrophil population and/or CD101⁻ refers to immature neutrophil population and/or
(in humans) CD11b⁺, CD101⁺, CD66b⁺, CD15⁺, CD101⁺, CD16^{+/-}, CD10^{+/-}, optionally wherein CD16^{low/-}CD10⁻ refers to immature neutrophil population, and/or CD16⁺CD10⁺ refers to the mature neutrophil population, and/or
(in RNAseq transcriptomic profiles) MMP8, MMP9, S100A9, S100A8, CSF3R, CXCR2.

In some embodiments, the method further comprises determining and/or measuring the pro-angiogenic ability of the cell, optionally, the method determines and/or measures markers comprising one or more of the following markers: MMP9, IL-6, VEGFa production, and/or combination thereof.

In some embodiments, the method further comprising sorting a plurality of cells into subpopulations, the method comprising clustering the heterogenous population into subpopulations based on their expression of one or more of the following:
(in mouse) Lin⁻, MHCII⁻, F4/80, Ly6C^{int}, Ly6G⁺, dcTRAIL-R1⁺, ARG-1⁺, VISTA⁺, and/or
(in human) CD11b⁺, CD101⁺, CD66b⁺, CD15⁺, TRAIL-R3⁺, ARG-1⁺, PTGS2⁺,
and the like.

In another aspect, there is provided a method of evaluating the progression of a proliferative disease in a subject, the method comprising:
determining/measuring an activity, an amount and/or a proportion of an immunosuppressive neutrophil in a first sample from the subject; and
comparing the activity, the amount and/or the proportion of an immunosuppressive neutrophil to those of a second sample from the subject, the second sample being obtained from subject at a later timepoint than the first sample,
wherein an increased activity, increased amount and/or increased proportion of an immunosuppressive neutrophil in the second sample as compared to the first sample is indicative of a worsening of the condition in the subject, and/or
wherein a reduced activity, reduced amount and/or reduced proportion of an immunosuppressive neutrophil in the second sample as compared to the first sample is indicative of an improvement of the condition in the subject.

In yet another aspect, there is provided a method of evaluating the efficacy of a treatment regimen (e.g. efficacy of a drug) for a proliferative disease in a subject, the method comprising:
determining/measuring an activity, an amount and/or a proportion of an immunosuppressive neutrophil in a first sample from the subject; and
comparing the activity, the amount and/or the proportion of an immunosuppressive neutrophil to those of a second sample from the subject, the second sample being obtained from subject at a later timepoint in the treatment regimen than the first sample,
wherein the treatment regimen is considered effective when the second sample has a reduced activity, reduced amount and/or reduced proportion of the immunosuppressive neutrophil as compared to the first sample and non-effective if otherwise.

In yet another aspect, there is provided a method of treating a proliferative disease in a subject, the method comprising:
administering to the subject a composition that is capable of decreasing an activity, an amount (or a level) and/or a proportion of an immunosuppressive neutrophil.

In some examples, the sample is a tissue biopsy, such as a tumour and/or cancer biopsy.

In yet another aspect, there is provided a kit for characterising an immunosuppressive neutrophil comprising one or more reagent that determines and/or measures the expression of one or more anti-apoptotic receptor markers comprising TRAIL-R3 and/or dcTRAILR1.

### Definitions

In various embodiments, the term “antibody” refers to an immunoglobulin molecule which specifically binds with an antigen. Antibodies can be intact immunoglobulins derived from natural sources or from recombinant sources and can be immunoreactive portions of intact immunoglobulins. Antibodies may be tetramers of immunoglobulin molecules. Tetramers may be naturally occurring or reconstructed from single chain antibodies or antibody fragments. Antibodies may also include dimers that may be naturally occurring or constructed from single chain antibodies or antibody fragments. The antibodies in the present invention may exist in a variety of forms including, for example, polyclonal antibodies, monoclonal antibodies, Fab and F(ab′)₂, as well as single chain antibodies (scFv), humanized antibodies, and human antibodies.

In various embodiments, the term “expression” refers to the transcription and/or translation of a particular nucleotide sequence driven by its promoter. In some embodiments, “expression” may refer to display of a polypeptide product of the transcription and/or translation of the nucleotide on the surface of a cell. Such polypeptides may be referred to as “cell surface markers” or “cell surface molecules.”

In various embodiments, the term “treating”, “treat” and “therapy”, and synonyms thereof refer to both therapeutic treatment and prophylactic or preventative measures, wherein the object is to prevent or slow down (lessen) a medical condition, which includes but is not limited to diseases (such as proliferative disease including tumour and/or cancer), symptoms and disorders. A medical condition also includes a body's response to a disease or disorder, e.g. dysregulated cell proliferation, dysregulated cell metabolism, and/or inflammation. Those in need of such treatment include those already with a medical condition as well as those prone to getting the medical condition or those in whom a medical condition is to be prevented.

In various embodiments, the term “subject” as used herein includes patients and non-patients. The term “patient” refers to individuals suffering or are likely to suffer from a medical condition such as proliferative disease including tumour and/or cancer, while “non-patients” refer to individuals not suffering and are likely to not suffer from the medical condition. “Non-patients” include healthy individuals, non-diseased individuals and/or an individual free from the medical condition. The term “subject” includes humans and animals. Animals may include, but is not limited to, mammals (for example non-human primates, canine, murine and the like), and the like. “Murine” refers to any mammal from the family Muridae, such as mouse, rat, rabbit, and the like.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Tumour infiltrating neutrophils make up a substantial fraction of the immune population in solid tumours, where they are re-programmed to acquire immunosuppressive abilities that promote tumour growth and progression. These immunosuppressive neutrophils are often termed polymorphonuclear myeloid derived suppressor cells (PMN-MDSCs).

PMN-MDSCs play important roles in promoting cancer growth, progression and eventual metastasis, and make up a significant population within the immunosuppressive tumour microenvironment (TME). Unlike their normal circulating neutrophil counterparts, PMN-MDSCs are involved in several tumour promoting processes: 1) acquisition of a immunosuppressive phenotype as exemplified by the expression of Arg1, PD-L1 and other inhibitory molecules, 2) remodelling of the TME and angiogenesis via secretion of MMPs and pro-angiogenic factors such as IL-6 and VEGFa, 3) co-opting reactive oxygen (ROS) and nitrogen species (RNS) pathways which can have immunosuppressive effects on anti-tumour effector cells, besides enhancing tumorigenesis. However, since both PMN-MDSCs and normal circulating neutrophils are both defined as Gr-1^{hi}CD11b⁺Ly6G⁺ cells in mice and CD11b⁺CD66b^{hi}CD15⁺ in humans, the differences between both populations in the tumour-bearing state remain poorly defined. Additionally, because there is no specific marker that defines PMN-MDSCs, this has hampered the study of this population in both mouse pre-clinical models and in human patients.

In view of the above, the inventors of the present disclosure identified the need to provide cell surface markers that distinguish these PMN-MDSCs from normal circulating neutrophils in both mouse and human. The inventors of the present disclosure believed that the characterisation of cell surface markers of PMN-MDSCs would advantageously improve functional studies and specific targeting of these cells for therapeutic purposes.

As such, exemplary, non-limiting embodiments of a method of detecting and/or sorting and/or characterising an immunosuppressive immune cell and further methods of using the immunosuppressive immune cells in therapy or diagnosis are disclosed hereinafter.

In various embodiments, there is provided a method of detecting and/or sorting and/or characterising an immunosuppressive immune cell comprising determining and/or measuring the expression of one or more anti-apoptotic gene/transcript/protein/marker/receptor, optionally the method determines and/or measures the expression of one or more anti-apoptotic receptors/gene/transcript/protein/marker.

In some examples, characterising an immune cell/immune cell population may comprise identifying a nature and/or a property associated with the immune cell/immune cell population. In some examples, characterising the immune cell/immune cell population comprises subsetting the populations based on one or more of their phenotypes, ontogenies, gene expression profiles, transcriptomic profiles, protein expression profiles and specialized functions. In some examples, characterising the immune cell/immune cell population comprises determining a type (including a subtype), a subpopulation, an expression profile, and/or a property (e.g., an ability/capability and/or propensity to induce/promote or modulate inflammation (i.e., activate or suppress one or more immune cells)) associated with the immune cell/immune cell population. In some examples, the immunosuppressive immune cell is an immunosuppressive neutrophil, optionally the immunosuppressive immune cell is a polymorphonuclear myeloid-derived suppressor cell (PMN-MDSC).

In one aspect, there is provided a method of detecting and/or characterising an immunosuppressive neutrophil comprising determining and/or measuring the expression of one or more anti-apoptotic markers on a neutrophil population.

In some embodiments, the neutrophil is a non-circulating neutrophil. In some examples, a neutrophil is a sub-population of the immune system that is generally recognised to express Gr-1^{hi}CD11 b⁺Ly6G⁺ (in mice) and/or CD11b⁺CD66b^{hi}CD15⁺ (in humans). In some embodiments, a precursor neutrophil is generally recognised or identified as Lin⁻CD115⁻Ly6C^{low}Siglec-F⁻Gr1⁺CD11b⁺CXCR4^{hi}ckit^{int}. In some embodiments, an immature neutrophil is generally recognised or identified as Lin⁻CD115⁻Ly6C^{low}Siglec-F⁻Gr1⁺CD11b+Ly6G⁺CD101⁻. In some embodiments, a mature neutrophil is generally recognised or identified as Lin⁻CD115⁻Ly6C^{low}Siglec-F⁻Gr1⁺CD11b⁺Ly6G⁺CD101⁺. In some embodiments, the neutrophil is part of a heterogenous neutrophil population including an effector neutrophil and an immunosuppressive neutrophil. In various embodiments, “effector neutrophil” refers to a sub-population of neutrophils that retain the natural ability to be activated, may begin to divide and/or secrete molecules (e.g., cytokines) that increase or upregulate an immune response towards a pathogen or an aberrant cell (such as a tumour or cancer cell). Thus, an effector neutrophil reduces and/or abates tumorigenesis. In some examples, the immunosuppressive neutrophil as described herein expresses Arg1, PD-L1, and other inhibitory molecules. In some examples, the immunosuppressive neutrophil as described herein remodels tumour microenvironment and angiogenesis via secretion of MMPs and pro-angiogenic factors such as IL-6 and VEGFa. In some examples, the immunosuppressive neutrophil as described herein co-opt reactive oxygen (ROS) and nitrogen species (RNS) pathways that can have immunosuppressive effect on anti-tumour effectors cells, thus enhancing tumorigenesis.

In various embodiments, the immunosuppressive neutrophil is a polymorphonuclear myeloid-derived suppressor cell (PMN-MDSC).

As shown in the Experimental Section, by utilizing transcriptomic and multi-parameter flow cytometric analysis in an orthotopic pancreatic cancer mouse model that recapitulates clinical progression of human pancreatic ductal adenocarcinoma (PDAC), the inventors of the present disclosure identified decoy TNF-related apoptosis-inducing ligand (TRAIL) receptor 1, (dcTRAILR1) as a candidate marker for PMN-MDSCs. dcTRAILR1 expression was specifically and highly induced on a subset of mature LyG+CD101+ neutrophils infiltrating the tumour, and was not expressed in neutrophils in the blood, spleen or the bone marrow of tumour-bearing mice. Expression of dcTRAILR1 in mature neutrophils was highly correlated with expression of PD-L1 (CD274) and VISTA among other immunosuppressive markers. These genes are well known to participate in immunosuppression in the tumour microenvironment and consequentially enable tumour immune evasion. Thus, this shows the dcTRAILR1+ tumour mature neutrophil subset is a bona fide PMN-MDSC population. Single-cell RNA sequencing (scRNAseq) of neutrophils from the tumour and subsequent RNA velocity analysis demonstrate that dcTRAILR1 identifies neutrophils that have the strongest upregulation of a PMN-MDSC transcriptional signature.

As such, in some examples, the method as described herein determines and/or measures the expression of one or more anti-apoptotic markers including, but not limited to TNF-related apoptosis-inducing ligand (TRAIL)-receptor 3 (TRAIL-R3/TNFRSF10C; in human) and/or decoy TNF-related apoptosis-inducing ligand (TRAIL)-receptor (dcTRAILR1; in mouse).

In some examples, the anti-apoptotic gene/transcript/protein/marker/receptor is TNF-related apoptosis-inducing ligand (TRAIL)-receptor 3 (TRAIL-R3/TNFRSF10C; human) and/or decoy TNF-related apoptosis-inducing ligand (TRAIL)-receptor (dcTRAILR1; mouse).

In some examples, the method further comprises: determining and/or measuring the expression of one or more genes/transcript/protein/receptor/marker (such as a surface marker) related and/or capable of inducing immunosuppression of immune cells (such as effector immune cells, including but not limited to, T cells, such as effector T cells). In some examples, the method further determines and/or measures the expression of one or more genes/transcripts/proteins/receptors/markers that induce immunosuppression of immune cells. In some examples, the method further comprises determining and/or measuring the expression of one or more markers related and/or capable of inducing immunosuppression of an immune cell.

As used herein, an immune cell includes but is not limited to an effector immune cell such as a lymphocyte and a macrophage. In some examples, an immune cell may be an effector immune such as T lymphocytes, B lymphocytes, and the like. In some examples, the immunosuppression induced by the immunosuppressive neutrophils as described herein may be immune effector cell such as effector T cells.

In some examples, the gene/transcript/protein/marker/receptor related and/or capable of inducing immunosuppression of effector immune cells includes, but is not limited to, CD274 (PD-L1), VSIR (VISTA), LILRB4, ARG1, PTGS2 (COX2), NOS2, and the like.

In various embodiments, the method comprises determining or measuring the expression of at least about 2, at least about 3, at least about 4, at least about 5, or at least about 6 of the genes/transcripts/proteins/markers/receptors that induce immunosuppression of an effector immune cell.

In some examples, the method further comprises:
determining and/or measuring the expression of one or more gene/transcript/protein/marker/receptor that negatively regulate inflammatory function, optionally negatively regulate innate inflammatory function.

In some examples, the gene/transcript/protein/marker/receptor that negatively regulate innate inflammatory function includes, but is not limited to, Dcir2, Pir-a/b, Clec12a, and the like. In various embodiments, the method comprises determining or measuring the expression of at least about 2, or 3 of the genes/transcripts/proteins/markers/receptors that negatively regulate innate inflammatory function.

In some examples, the method further comprises:
determining and/or measuring the expression of one or more gene/transcript/protein/marker/receptor of metabolic ectoenzymes that create immunosuppressive tumour microenvironment (TME). In some examples, wherein the gene/transcript/protein/marker/receptor of metabolic ectoenzymes that create the immunosuppressive TME includes, but is not limited to, CD39, CD73, and the like, and/or combinations thereof.

In various embodiments, the method comprises determining or measuring the expression of at least about 2, at least about 3, of the genes/transcripts/proteins/markers/receptors of metabolic ectoenzymes that create immunosuppressive tumour microenvironment.

In some examples, the method further comprises:
determining and/or measuring the expression of one or more gene/transcript/protein/marker including, but is not limited to, (in mouse) CD11b⁺, Gr-1^{hi}, Ly6C^{low}, Ly6G⁺, CD101^{+/-}, optionally wherein CD101⁺ refers to mature neutrophil population and/or CD101⁻ refers to immature neutrophil population and/or
(in humans) CD11b⁺, CD101⁺, CD66b⁺, CD15⁺, CD101⁺, CD16^{+/-}, CD10^{+/-}, optionally wherein CD16^{low/-}CD10⁻ refers to immature neutrophil population, and/or CD16⁺CD10⁺ refers to the mature neutrophil population, and/or
(in RNAseq transcriptomic profiles) MMP8, MMP9, S100A9, S100A8, CSF3R, CXCR2.

In various embodiments, the method comprises determining or measuring the expression of at least about 2, at least about 3, at least about 4, at least about 5, or at least about 6, or at least about 7, or at least about 8, or at least about 9, or at least about 10, or at least about 11, or at least about 12, or at least about 13, or at least about 14, or at least about 15 of the genes/transcripts/proteins/markers/receptors that refers/characterise a mature neutrophil population and/or an immature neutrophil population.

In some examples, the method further comprises
determining and/or measuring the pro-angiogenic ability of the cell, optionally, the method determines and/or measures one or more of MMP9, IL-6, VEGFa production, and/or combination thereof.

In various embodiments, the method comprises determining or measuring the expression of at least about 2, at least about 3, at least about 4 of the genes/transcripts/proteins/markers/receptors that shows the pro-angiogenic ability of a cell.

In some examples, wherein the determining and/or measuring of the expression of one or more gene/transcripts/proteins/markers comprises clustering the heterogenous population into subpopulations based on their protein expression profiles and/or transcriptome profiles.

In some examples, the method further comprises performing RNA sequencing (RNA-seq) to obtain the gene expression profiles/transcriptome profile. In various examples, the gene expression profiles/transcriptome profiles are obtained by deep sequencing. In some examples, the method further comprises high dimensional sequencing techniques to obtain transcriptional and/or epigenetic profile of PMN-MDSCs. In some examples, the method further comprises single-cell RNAseq, single-cell ATACseq, and the like.

In some examples, wherein the method further comprising performing flow cytometry, mass cytometry/Cytometry by Time-Of-Flight (CyTOF) mass spectrometry and/or immunostaining to obtain the gene expression profiles/protein expression profiles.

In some examples, the method further comprises performing flow cytometry to obtain protein expression profiles.

In some examples, the method further comprises index sorting individual cells based on their known characteristics such as one or more of the following characteristics: defined size, granularity and selected marker expressions.

The gene expression profiles/protein expression profiles may also be determined/measured by other methods known in the art, such as Western Blotting, immunofluorescence (IF) microscopy, immunohistochemistry (IHC), immunocytochemistry, enzyme-linked immunosorbent assay (ELISA), and the like.

In some examples, wherein the method further comprising sorting/detecting/characterising a plurality of cells/a population of cell into subpopulations, the method comprising clustering the heterogenous population into subpopulations based on their expression of one or more of the following genes/transcripts/proteins:
(in mouse) Lin⁻, MHCII⁻, F4/80⁻, Ly6Ci⁺, Ly6G⁺, dcTRAIL-R1⁺, ARG-1⁺, VISTA⁺, and/or
(in human) CD11b⁺, CD101⁺, CD66b⁺, CD15⁺, TRAIL-R3⁺, ARG-1⁺, PTGS2⁺,
and the like.

In various embodiments, the method comprises determining or measuring the expression of at least about 2, at least about 3, at least about 4, at least about 5, or at least about 6, or at least about 7, or at least about 8 of the genes/transcripts/proteins/markers/receptors that refer/characterise a plurality of cells/a population of cell into subpopulations.

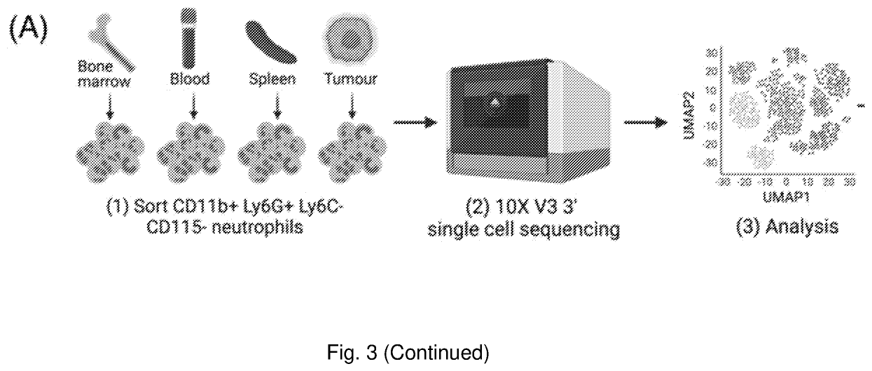

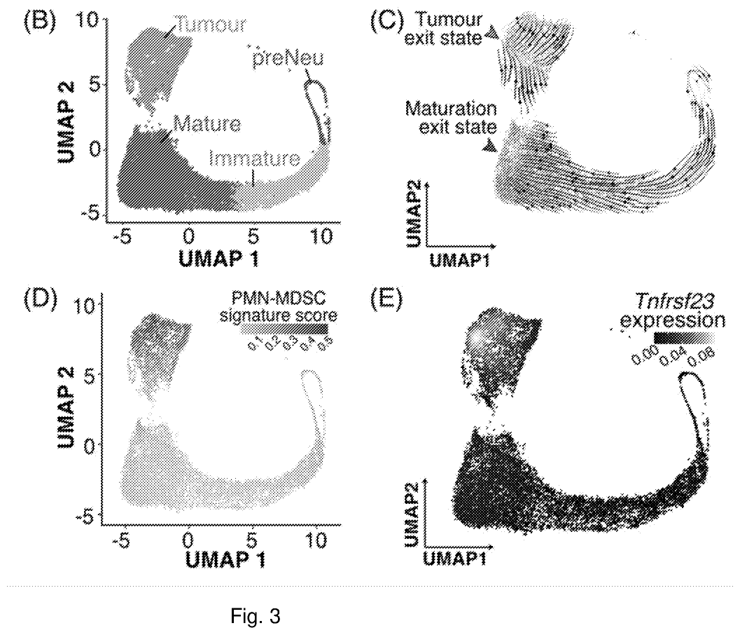

At the same time, as disclosed in the Experimental Section, further correlation studies with the PRECOG (Prediction of Clinical Outcomes from Genomic data) dataset (Stanford) indicate that high expression of the human decoy receptor ortholog, TRAIL-R3 (FIG. 3), is correlated with negative survival outcomes in human pancreatic cancer. Taken together, based on the discovery from the preclinical model as described herein, the utility of TRAIL-R3 as a theragnostic marker to identify and target PMN-MDSCs for depletion in cancer was demonstrated by the inventors of the present disclosure.

Therefore, in various embodiments, there is provided a method of identifying/determining/diagnosing a condition in a subject, the method comprising:
determining/measuring a presence, an activity, an amount and/or a proportion of an immunosuppressive immune cell (such as an immunosuppressive neutrophil/PMN-MDSC) in a sample from the subject,
wherein the presence of, a high activity of, a high amount of and/or a high proportion of an immunosuppressive immune cell (such as an immunosuppressive neutrophil/PMN-MDSC) in the sample is indicative of a presence of, a risk of or severity to the condition in the subject,
wherein the condition is a proliferative disease (such as tumour and/or cancer).

In various embodiments, there is provided a method of determining/evaluating progression of a condition in a subject, the method comprising:
determining/measuring an activity, an amount and/or a proportion of an immunosuppressive immune cell (such as an immunosuppressive neutrophil/PMN-MDSC) in first sample from the subject; and
comparing the activity, the amount and/or the proportion of an immunosuppressive immune cell (such as an immunosuppressive neutrophil/PMN-MDSC) to those of a second sample from the subject, the second sample being obtained from subject at a later timepoint than the first sample,
wherein an increased activity, increased amount and/or increased proportion of an immunosuppressive immune cell (such as an immunosuppressive neutrophil/PMN-MDSC) in the second sample as compared to the first sample is indicative of a worsening of the condition in the subject,
wherein a reduced activity, reduced amount and/or reduced proportion of an immunosuppressive immune cell (such as an immunosuppressive neutrophil/PMN-MDSC) in the second sample as compared to the first sample is indicative of an improvement of the condition in the subject, and
wherein a similar activity, similar amount and/or similar proportion of an immunosuppressive immune cell (such as an immunosuppressive neutrophil/PMN-MDSC) in the first sample as compared to the second sample is indicative of a non-improvement and/or stabilisation of the condition in the subject,
wherein the condition is a proliferative disease (such as tumour and/or cancer).

In another aspect of the present disclosure, there is provided a method of evaluating the progression of a proliferative disease in a subject, the method comprising:
determining/measuring an activity, an amount and/or a proportion of an immunosuppressive neutrophil in a first sample from the subject; and
comparing the activity, the amount and/or the proportion of an immunosuppressive neutrophil to those of a second sample from the subject, the second sample being obtained from subject at a later timepoint than the first sample,
wherein an increased activity, increased amount and/or increased proportion of an immunosuppressive neutrophil in the second sample as compared to the first sample is indicative of a worsening of the condition in the subject, and/or
wherein a reduced activity, reduced amount and/or reduced proportion of an immunosuppressive neutrophil in the second sample as compared to the first sample is indicative of an improvement of the condition in the subject.

Also contemplated in various embodiments is a method of determining/evaluating the efficacy of a treatment regimen (e.g., efficacy of a drug) for a condition in a subject, the method comprising:
determining/measuring an activity, an amount and/or a proportion of an immunosuppressive immune cell (such as an immunosuppressive neutrophil/PMN-MDSC) in first sample from the subject; and
comparing the activity, the amount and/or the proportion of an immunosuppressive immune cell (such as an immunosuppressive neutrophil/PMN-MDSC) to those of a second sample from the subject, the second sample being obtained from subject at a later timepoint in the treatment regimen than the first sample,
wherein the treatment regimen is considered effective when the second sample has a reduced activity, reduced amount and/or reduced proportion of an immunosuppressive immune cell (such as an immunosuppressive neutrophil/PMN-MDSC) as compared to the first sample and non-effective if otherwise
wherein the condition is a proliferative disease (such as tumour and/or cancer).

In yet another aspect of the present disclosure, there is provided a method of evaluating the efficacy of a treatment regimen (e.g. efficacy of a drug) for a proliferative disease in a subject, the method comprising:
determining/measuring an activity, an amount and/or a proportion of an immunosuppressive neutrophil in a first sample from the subject; and
comparing the activity, the amount and/or the proportion of an immunosuppressive neutrophil to those of a second sample from the subject, the second sample being obtained from subject at a later timepoint in the treatment regimen than the first sample,
wherein the treatment regimen is considered effective when the second sample has a reduced activity, reduced amount and/or reduced proportion of the immunosuppressive neutrophil as compared to the first sample and non-effective if otherwise.

In various embodiments, there is provided a method of slowing down/retarding/arresting/treating a proliferative disease (such as tumour and/or cancer) in a subject, the method comprising:
administering to the subject an agent/composition that is capable of modulating/reducing/eliminating/depleting an activity, an amount (or a level) and/or a proportion of an immunosuppressive immune cell (such as an immunosuppressive neutrophil/PMN-MDSC).

In yet another aspect of the present disclosure, there is provided a method of treating a proliferative disease in a subject, the method comprising: administering to the subject a composition that is capable of decreasing an activity, an amount (or a level) and/or a proportion of an immunosuppressive neutrophil.

In various embodiments, there is provided an agent/composition that is capable of modulating/reducing/eliminating/depleting an activity, an amount (or a level) and/or a proportion of an immunosuppressive immune cell (such as an immunosuppressive neutrophil/PMN-MDSC) for use in therapy. In various embodiments, there is provided an agent/composition that is capable of modulating/reducing/eliminating/depleting an activity, an amount (or a level) and/or a proportion of an immunosuppressive immune cell (such as an immunosuppressive neutrophil/PMN-MDSC) for use in slowing down/retarding/arresting/treating a proliferative disease (such as cancer and/or tumour).

In various embodiments, the agent/composition comprises inhibitors and/or activators of one or more genes/transcripts/proteins/phenotypes expressed by or associated with an immunosuppressive immune cell (such as an immunosuppressive neutrophil/PMN-MDSC). In various embodiments, the agent/composition comprises a binding molecule of an immunosuppressive immune cell (such as an immunosuppressive neutrophil/PMN-MDSC), optionally wherein the binding molecule is capable of modulating/reducing/eliminating/depleting an activity, an amount (or a level) and/or a proportion of an immunosuppressive immune cell (such as an immunosuppressive neutrophil/PMN-MDSC). An immunosuppressive immune cell (such as an immunosuppressive neutrophil/PMN-MDSC)-binding molecule may be identified by screening of chemical compound libraries or small molecules libraries and/or antibody libraries and/or suitable assays such as ELISA. In one example, the agent/composition comprises anti-TRAIL-R3 (or dcTRAIL-R1) antibody. In various embodiments, the agent/composition comprises a selective agent/composition that targets TRAIL-R3 (or dcTRAIL-R1). Advantageously, such agents/compositions do not target inflammatory neutrophils (i.e., neutrophils that do not express TRAIL-R3 and/or dcTRAIL-R1) and its immune functions (such as pro-inflammatory), which may lead to an improvement in patient outcome. The agent/composition may also be administered as an adjuvant therapy or as part of a combination therapy.

In various embodiments, the agent/composition may be an antibody. For example, the agent/composition may be a depleting and/or neutralising antibody against cell surface marker dcTRAILR1 and/or TRAIL-R3.

In some examples, as used herein, the term “sample” refers to a tissue biopsy, such as a tumour and/or cancer biopsy.

In some examples, wherein the immunosuppressive immune cell is obtained/provided/isolated/found in a sample of a subject.

In some examples, the subject may be a mammal. In some examples, the subject may be human, non-human primate, rodent (such as mouse), and the like.

In some examples, the subject may be a patient suspected of having and/or determined to have proliferative disease. In some examples, the subject may be suspected of having a tumour and/or cancer. In some examples, the subject be suspected and/or have a solid tumour.

In some examples, the solid tumour may include, but is not limited to, pancreatic tumour/cancer, lung tumour/cancer (such as non-small cell lung cancer/NSCLC and/or small cell lung cancer/SCLC), sarcoma tumour/cancer (such as Ewing and/or osteosarcoma), head and neck tumour/cancer (such as oesophageal, oral, and the like), gastric tumour/cancer, liver tumour/cancer, kidney tumour/cancer, germ cell tumour/cancer, colon tumour/cancer, breast tumour/cancer, adrenocortical tumour/cancer, melanoma (such as metastasised melanoma), ovarian tumour/cancer, bladder tumour/cancer, adrenocortical tumour/cancer, prostate tumour/cancer, and the like.

In some examples, the solid tumour may include, but is not limited to pancreatic tumour/cancer, sarcoma tumour/cancer (such as Ewing), lung tumour/cancer (such as squamous cell lung carcinoma/SCC, large cell carcinoma/LCC, small cell lung cancer/SCLC, and the like), gastric tumour/cancer, bladder tumour/cancer, adrenocortical tumour/cancer, prostate tumour/cancer, liver tumour/cancer, colon tumour/cancer, melanoma tumour/cancer, and the like.

In some examples, a kit for detecting and/or screening and/or characterising an immunosuppressive immune cell comprising one or more reagent that determines and/or measures the expression of gene/transcripts/proteins/markers/receptors as described herein.

In some examples, wherein the kit comprises
one or more reagent that determines and/or measures the expression of one or more anti-apoptotic gene/transcripts/proteins/markers/receptor, optionally the kit further comprises one or more reagent that determines and/or measures the expression of one or more anti-apoptotic receptor.

In yet another aspect, there is provided a kit for characterising an immunosuppressive neutrophil comprising one or more reagent that determines and/or measures the expression of one or more anti-apoptotic receptor markers comprising TRAIL-R3 and/or dcTRAILR1.

In some examples, the reagent may comprise antigen binding protein or antibody that recognises one or more anti-apoptotic receptor markers, optionally the marker is TRAIL-R3 and/or dcTRAILR1.

In some embodiments, there is provided the method or product as described herein.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

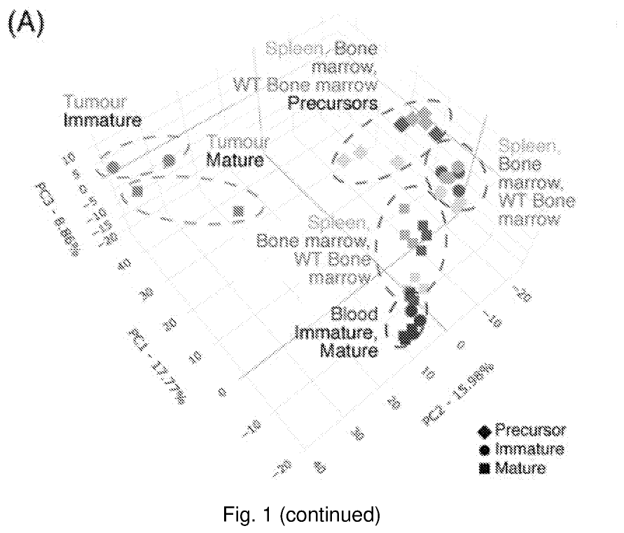

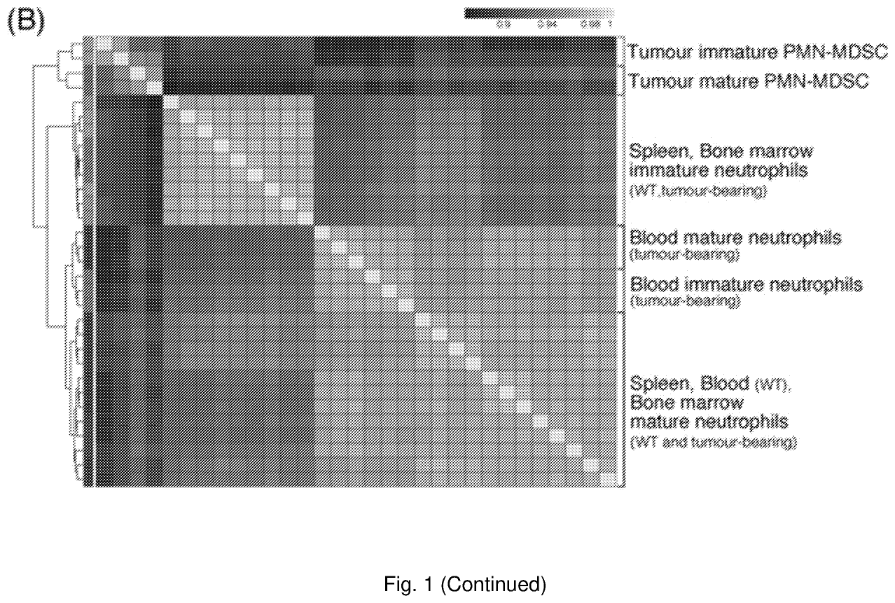

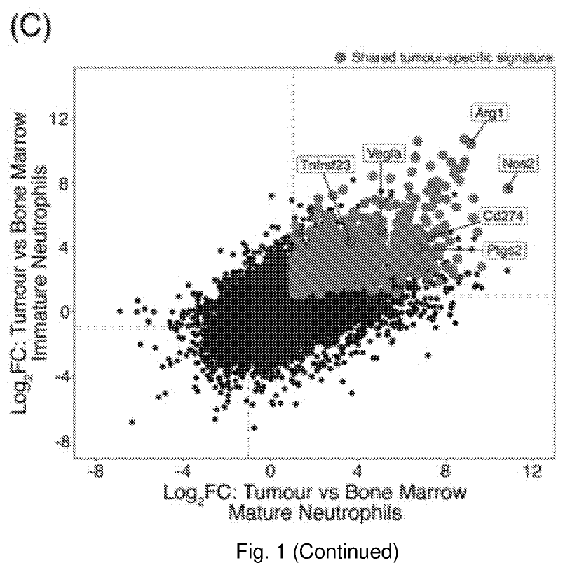

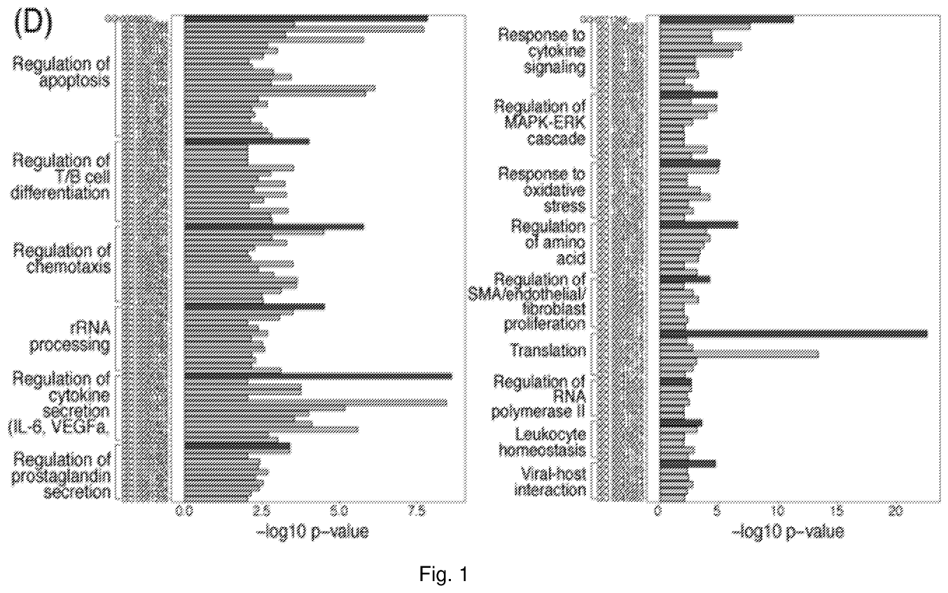

FIG. 1 shows tumour immature and mature neutrophil subsets are transcriptionally distinct from their equivalent neutrophil subsets from the blood, spleen or bone marrow. (A) Principal component analysis (PCA) of sorted precursor, immature and mature neutrophil subsets as labelled from tumour, spleen, blood and bone-marrow in wild-type (WT) or tumour bearing mice. (B) Spearman correlation heatmap of gene expression of all immature and mature neutrophil subsets analysed in (A). (C) Scatterplot showing Log2 fold change (Log2FC) values of differential gene analysis of tumour against bone-marrow immature (y-axis) and mature (x-axis) neutrophils. Shared tumour-specific genes across tumour immature and mature neutrophils are coloured in light grey, genes known to contribute to PMN-MDSC function are labelled: Tnfrsf23 (dcTrailr1), Arg1, Cd274 (Pd-I1), Ptgs2 (Cox2), Nos2 and Vegfa. (D) REVIGO analysis of shared tumour-specific genes shown as bar plots. Black bar illustrates the GO pathway within the cluster that has the lowest p-value.

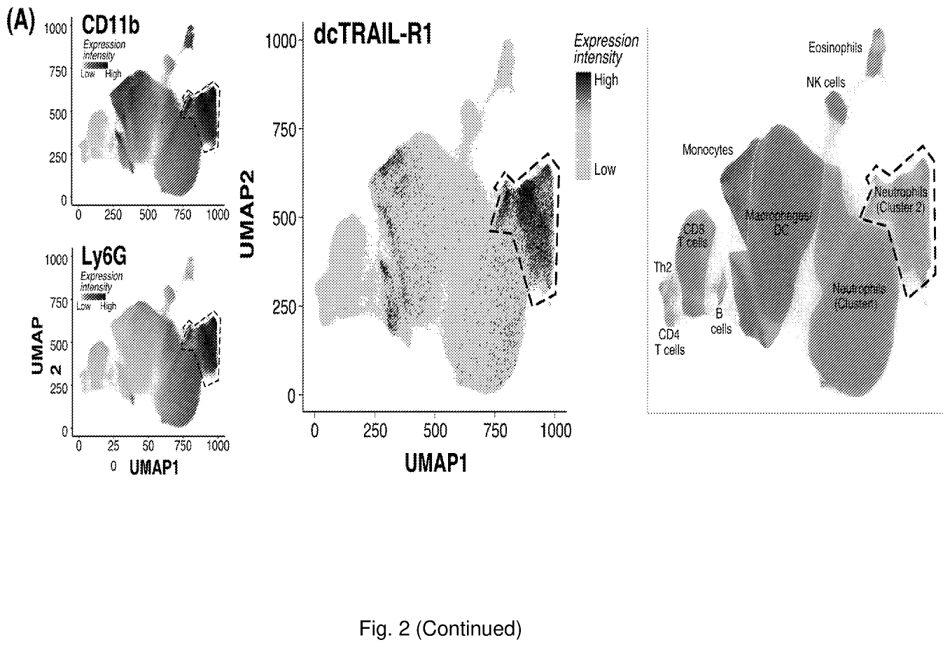

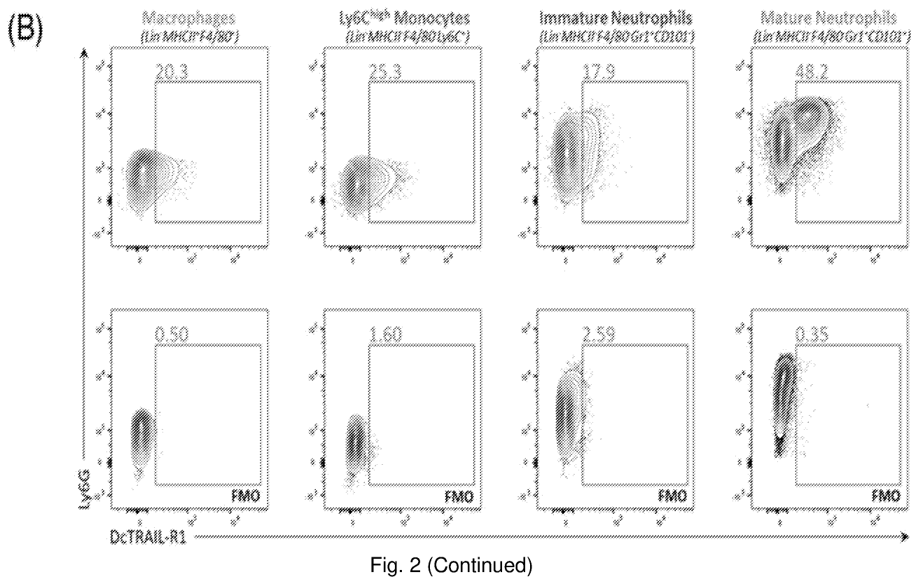

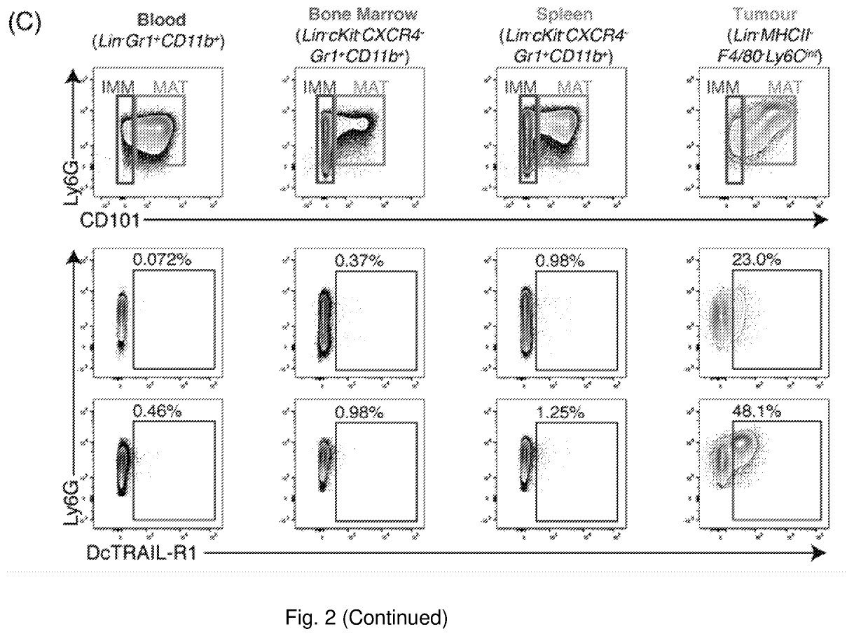

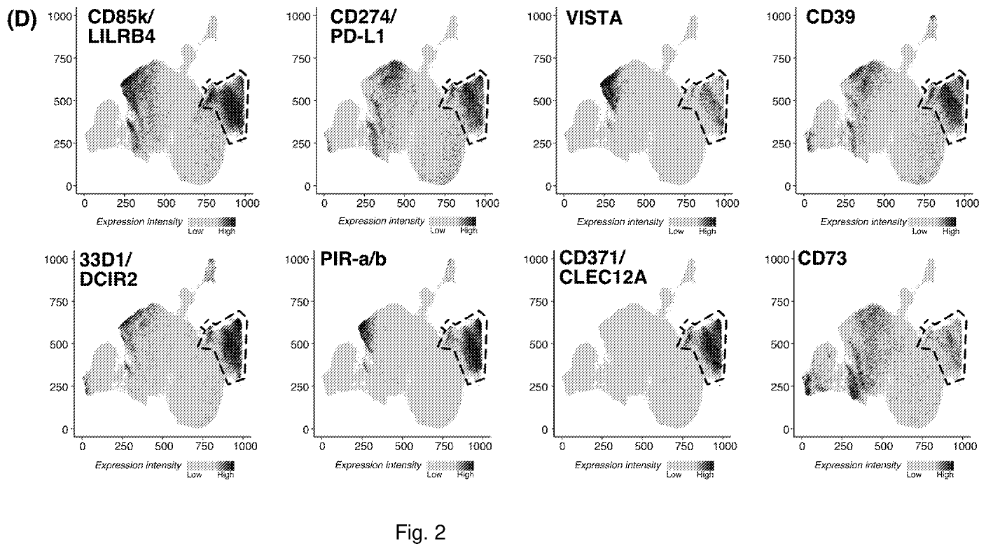

FIG. 2 shows DcTRAIL-R1 protein expression is specific only for a subset tumour-infiltrating mature neutrophils which co-expresses immunosuppressive and inhibitory receptors (on next page). (A) UMAP projection showing clustering of all CD45+ cells infiltrating the pancreatic adenocarcinoma (PDAC) tumour at 6 weeks. Log2MFI intensity of CD11b, Ly6G and DcTRAIL-R1 is shown from high (black) to low (grey) intensity. A separate plot indicating the corresponding cell type for each cluster is included on the left. Neutrophil cluster 2, which denotes the probable PMN-MDSC population is outlined in all UMAP projections. (B) Representative flow plots showing DcTRAIL-R1 expression for gated macrophages (Lin-MHCII+F4/80+), monocytic MDSC (Lin-MHCII-F4/80− Ly6C+), immature neutrophils (Lin-MHCII-F4/80-Gr− 1+CD101−) and mature neutrophils (Lin-MHCII-F4/80-Gr− 1+CD101+). Fluorescence minus one control is shown in the bottom row. (C) Top row: Representative flow plots showing CD101 and gating for expression for immature neutrophils (Lin-MHCII-F4/80-Gr-1+CD101−) and mature neutrophils (Lin-MHCII-F4/80-Gr-1+CD101+) in blood, bone marrow, spleen and tumour, Middle row: Representative flow plots showing DcTRAIL-R1 expression for immature neutrophils across labelled tissues, Last row: Representative flow plots showing DcTRAIL-R1 expression for mature neutrophils across labelled tissues. (D) UMAP projection representing measured log 2MFI intensity for labelled immunosuppressive/inhibitory markers are shown for all CD45+ cells.

FIG. 3 shows single cell RNA sequencing validates dcTRAILR1 expression as a marker of PMN-MDSCs in the tumour. (A) Schematic showing the workflow for single cell RNA sequencing (scRNAseq) in neutrophils from tumour bearing mice. (B) UMAP visualization of neutrophil clusters assigned based on organ of origin (bone marrow/spleen/blood/tumour) and maturation stage. Tumour neutrophils (labelled “Tumor”) separated clearly from neutrophils from the other organs, which instead grouped together according to maturation stage (labelled “preNeu”—precursor, labelled “immature”—immature, labelled “mature”—mature). (C) UMAP visualization of the velocity vector field showing the tumour and maturation exit states (arrowheads). RNA velocity analysis projected onto the UMAP visualization; velocity vector field denoted as streamlines with arrows showing direction of differentiation. (D) The PMN-MDSC signature is highly expressed in cells present in the tumour exit state. The PMN-MDSC signature derived from FIG. 1C was scored for each cell, and log-normalized expression is displayed. (E) Expression of dcTRAILR1 is highest in the tumour exit state. UMAP shows log-normalized expression of Tnfrsf23, the gene encoding for dcTRAILR1.

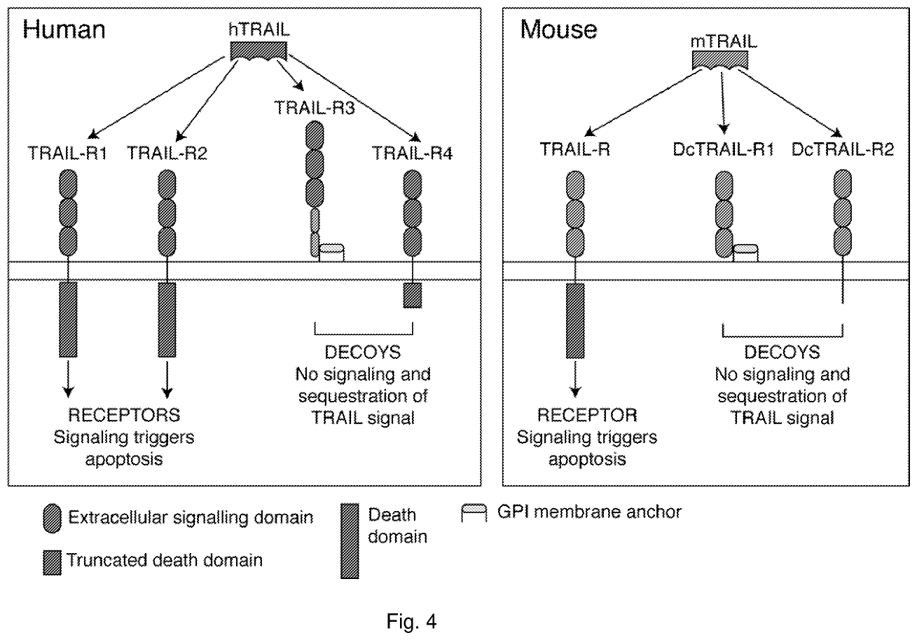

FIG. 4 shows TRAIL signalling pathway in the mouse and human. Depiction of human (hTRAIL, left) and mouse (mTRAIL, right) TRAIL signalling pathway. TRAIL binding to receptors TRAIL-R1 and TRAIL-R2 in human and TRAIL-R in mice results in receptor oligomerization and signalling from the oligomerized death domain complex to trigger cell apoptosis. The decoy receptors TRAIL-R3/R4 in humans, and DcTRAIL-R1/R2 in mice either do not have intracellular death domains or truncated ones, thus they are unable to signal to trigger downstream apoptosis.

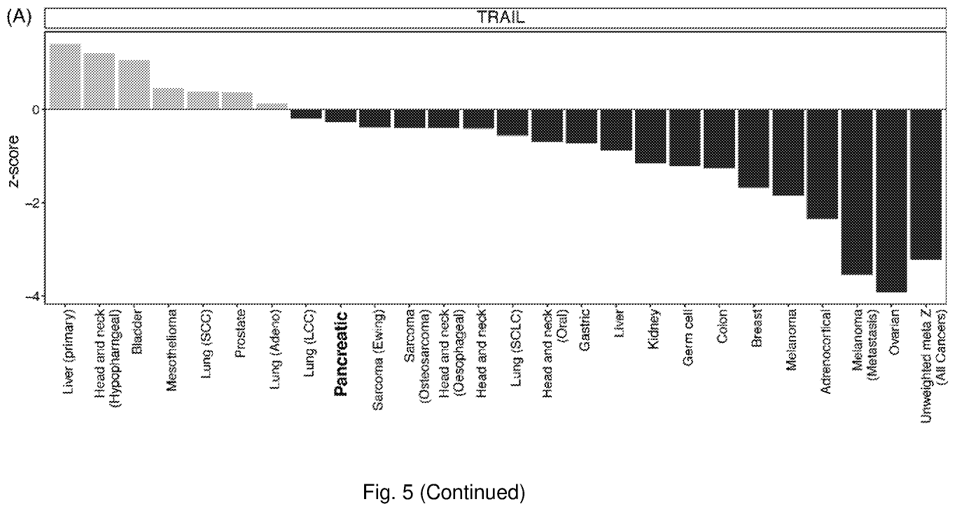

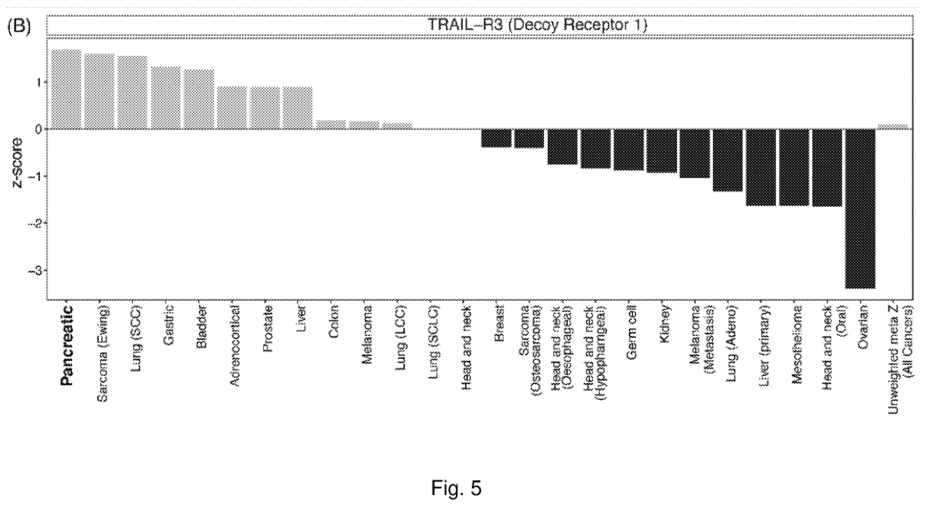

FIG. 5 shows TRAIL-R3 decoy receptor expression in human pancreatic cancer correlates with decreased overall survival. (A) Bar plots showing z-scores for individual cancer types obtained from PRECOG (Prediction of Clinical Outcomes based on Genomic data) dataset, where z scores are normalized across all cancers and correlate with increased adverse events (positive score, above z-score 0) or decreased adverse events (negative score, below z-score 0) dependent on their expression of TRAIL. Pancreatic cancer is highlighted in bold. (B) Bar plots showing z-scores for individual cancer types for overall survival dependent on their expression of TRAIL-R3 decoy receptor. Pancreatic cancer is highlighted in bold.

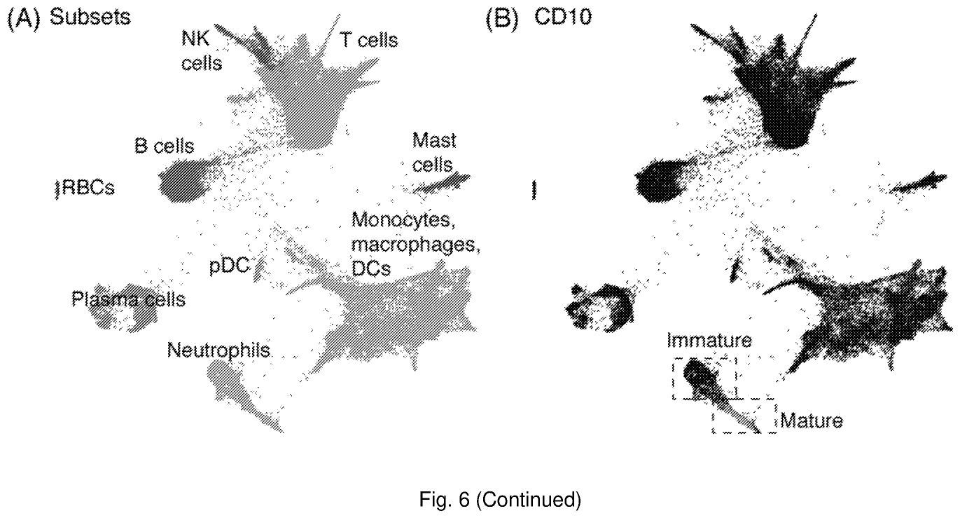

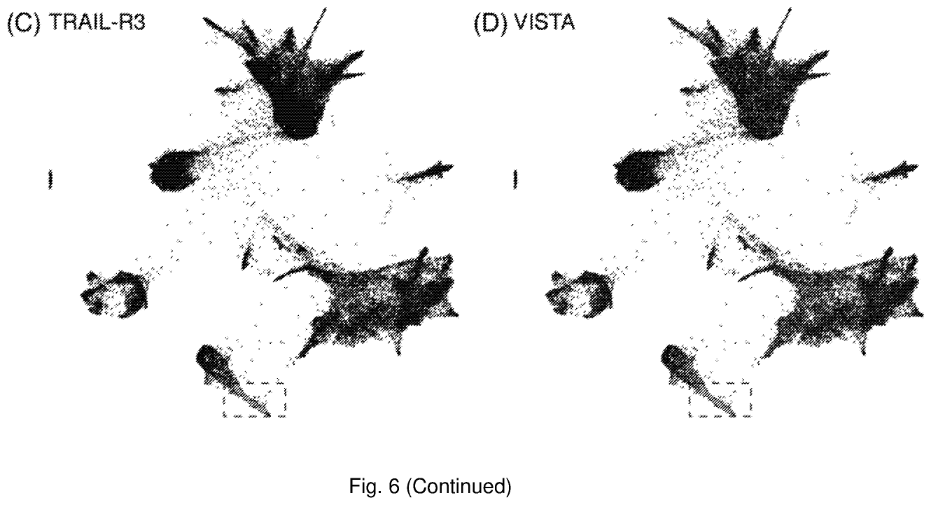

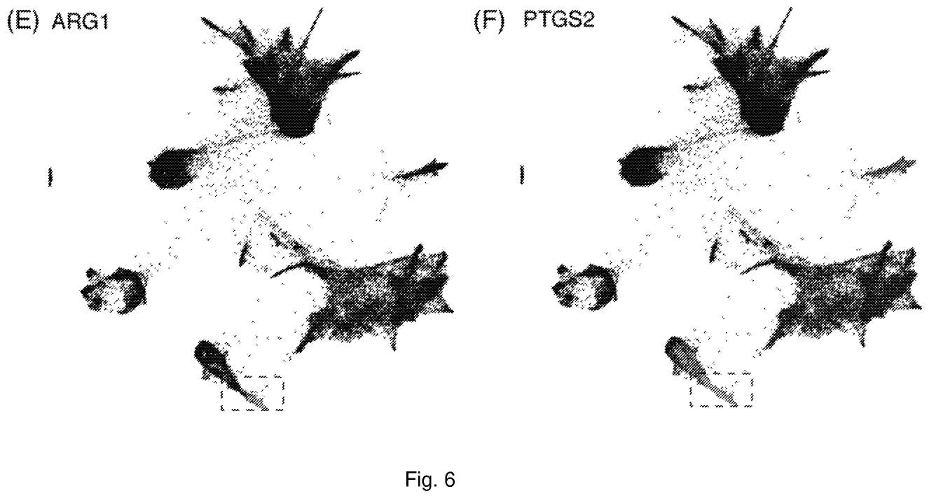

FIG. 6 shows TRAIL-R3 decoy receptor expression is expressed a subset of tumour-infiltrating mature neutrophils in human non-small cell lung cancer (NSCLC) (A) Two-dimensional visualization (SPRING plots) of immune cell transcriptomes derived from Zilionis et al. 2019. Individual cell types identified in the paper are coloured and labelled accordingly. Immature and mature neutrophils were defined according to CD10 expression, and populations are denoted in boxes as in (B). The rest of the following plots show gene expression across all immune subsets for (B) CD10 (MME), (C) TRAIL-R3 (TNFRSF10C), (D) VISTA (VSIR), (E) ARG1, (F) PTGS2 (COX2).

### EXPERIMENTAL SECTION

### Tissue Preparation and Data Analysis for Flow Cytometry and Cell Sorting

Blood was obtained through cardiac puncture and lysed in red blood cell (RBC) lysis buffer (eBioscience). For bone marrow cells, mice femurs were flushed with a 23-gauge needle in FACS buffer (i.e. PBS containing 2 mM EDTA and 2% foetal bovine serum (FBS)) and passed through a 70-μm nylon mesh sieve. Spleens were dissected and homogenized into single-cell-suspensions using a 70-μm nylon mesh, and subsequently lysed using RBC lysis buffer. Mice were administered with intrapancreatic injections of FC1242 tumour cells (kind gift from Dr Dannielle D. Engle, Tuveson lab) derived from Pdx1cre; LsL-KrasG12D/+; LsL−Trp53R172H/+(termed KPC) mice. The resulting orthotopic tumour was resected at week 6 following surgery and weighed. The tumour was minced into small pieces using surgical scissors, and digested in Digestion Buffer comprising RPMI-1640 (Gibco) containing 35 U/ml of Collagenase IV (Sigma-Aldrich), 0.25 mg/ml of DNAse I (Roche Life Sciences), and 10% FBS for 30 minutes. The volume of digestion buffer utilized was 1 ml per 0.5 g of tumour tissue. After digestion, tumours were further dispersed in GentleMACS™ 50 M tubes for 40 s and filtered through 70-μm nylon mesh sieves. The single cell suspension was washed with FACS buffer and subsequently lysed with RBC lysis buffer. For identification of immature and mature neutrophil subsets, cells suspensions were stained with fluorophore-conjugated anti-mouse antibodies against CD11b (M1/70), CD45 (30-F11), CD101 (Moushi101), CD115 (AFS598), CX3CR1 (SA011F11), F4/80 (BM8), Gr1 (RB6-8C5 Ly6C (HK1.4), Ly6G (1A8), Siglec-F (E50-2440), CXCR4 (2B11) and cKit (2B8) together with exclusion lineage markers that include CD3e (145-2C11), CD90.2 (53-2.1), B220 (RA3-6B2), NK.1.1 (PK136), CD11c (N418), and MHC-IA/E (M5/114.15.2)). After exclusion of cell doublets and dead cells by DAPI, precursor neutrophils were identified Lin⁻CD115⁻ Ly6C^{low}Siglec-F⁻Gr1⁺CD11b⁺CXCR4^{hi}ckit^{int}, immature neutrophils were identified as Lin⁻ CD115⁻Ly6C^{low}Siglec-F⁻Gr1⁺CD11b⁺Ly6G⁺CD101⁻, while mature neutrophils were identified as Lin⁻CD115⁻Ly6C^{low}Siglec-F⁻Gr1⁺CD11b⁺Ly6G⁺CD101⁺. For validation of dcTRAIL-R1 expression, cells were stained with biotin-conjugated dcTRAIL-R1 (BAM2378), and subsequently stained with fluorophore-conjugated streptavidin secondary antibodies. Flow cytometry acquisition was performed on a 5-laser BD LSR II™ (BD) using FACSDiva™ software, and data was subsequently analysed with FlowJo™ software (Tree Star). Sorting of neutrophil subsets were performed using a BD ARIA II™ (BD) to achieve >98% purity.

Bulk RNA Sequencing

For bulk RNA sequencing, precursor, immature, and mature neutrophils were sorted as detailed above. Total RNA was extracted using Arcturus PicoPure™ RNA Isolation kit (Applied Biosystems Thermo Fisher Scientific) according to manufacturer's protocol. All RNAs were analysed on Agilent Bioanalyser for quality assessment with a median RNA Integrity Number (RIN) of 9.4. cDNA libraries were prepared using 2 ng of total RNA with 1 μl of 1:50,000 dilution of ERCC RNA Spike in Controls respectively (Ambion Thermo Fisher Scientific) using the SMART-Seq v2 protocol (Picelli et al., 2014, Full-length RNA-seq from single cells using Smart-seq2, Nature Protocols, Vol. 9, No. 1, pages 171 to 181, the content of which is incorporated herein in its entirety) with the following modifications: 1. Addition of 20 mM TSO; 2. Use of 200 pg cDNA with 1/5 reaction of Illumina Nextera XT kit (Illumina, San Diego, CA, USA). The length distribution of the cDNA libraries was monitored using a DNA High Sensitivity Reagent Kit on the Perkin Elmer Labchip (Perkin Elmer, Waltham, MA, USA). All samples were subjected to an indexed paired-end sequencing run of 2×151 cycles on an Illumina HiSeq 4000 system (Illumina) (26 samples/lane). Raw reads were aligned to mouse genome build GRCm38 using STAR aligner. Read counts per gene were then calculated using the feature Counts (part of the Subread package) based on GENCODE gene annotation version M20. Log 2 transformed reads per kilobase per million mapped reads (log 2 RPKM) normalization was done to account for transcript length and the total number of reads. Differentially expressed genes (DEGs) analysis was done using DESEq2 on protein coding genes only. DEGs with FDR (False Discovery Rate) less than 0.05 were selected as statistically significant. PCA analysis was performed using the prcomp base command in R (4.0.2), gene ontology performed using the topGO package (2.42.0), and clustering of gene ontology results by REVIGO (http://revigo.irb.hr/).

LEGENDScreen and InfinityFlow Pipeline

Pancreatic tumours were harvested as detailed above from 10 tumour-bearing mice 6 weeks post injection. Mouse cells were first stained a backbone panel cocktail of mouse antibodies to define the various immune lineages in the tumour. These markers include: CD11b (M1/70), Ly6C (HK1.4), D43 (S11), Ly6G (1A8), CD45 (30-F11), CD101 (Moushi101), Siglec-F (E50-2440), Gr1 (RB6-8C5), MHC II (M5/114.15.2). CD43 (S7), and lineage markers (CD90.2 (53-2.1), B220 (RA3-6B2), NK.1.1 (PK136)) and CD11c (Clone N418). After 60 min of staining at 4° C., cells were washed in FACS buffer, then spun down at 400 g for 5 min. Cells were equally aliquoted by volume into individual wells containing specific PE-conjugated markers in the Mouse PE LEGENDScreen™ kit (Biolegend). After staining for 60 mins, plates were washed and before flow cytometry acquisition was performed on a 5-laser BD LSR II (BD) using FACSDiva™ software, and data was subsequently processed utilizing the Infinity Flow package in R (1.0.0.). The Infinity Flow pipeline involves regression analysis of the intensities of the PE-bound markers using the intensities of the back-bone markers. For each of the 261 FCS files obtained from the LEGEND Screen stain, each predicted marker intensity was transformed back to a linear intensity scale, concatenated with the backbone and the PE-marker expression values and exported back as a single FCS file, which can be analysed in FlowJo™ to identify distinct clusters.

Single Cell RNA Sequencing (scRNAseq)

Two biological replicates of total neutrophil populations, identified as Lin⁻ CD45⁺CD115⁻Ly6C^{low}Siglec-F⁻Gr1⁺CD11b⁺Ly6G⁺, were sorted from the bone marrow, spleen, blood and tumours of two pancreatic tumour-bearing mice. Cells were spun down and incubated with TotalSeq™-A anti-mouse Hashtag antibodies 1-8 at a concentration of 0.5 μg per 100,000 cells for 30 mins at 4° C. Cells were washed with FACS buffer, spun down, and resuspended in PBS with 1% bovine serum albumin. Cells were then counted and pooled accordingly and were used as input for 10× Genomics 3′ (v3) sequencing following the manufacturer's protocol. The quality of sequencing reads was evaluated using FastQC and MultiQC. Cell Ranger (version 2.2.0) was used to align the sequencing reads (fastq) to the mm10 mouse transcriptome and quantify the expression of transcripts in each cell. This pipeline resulted in a gene expression matrix for each sample, which records the number of UMIs for each gene associated with each cell barcode. The gene expression matrix was subsequently analysed in R as a Seurat object using the Seurat package (4.0.5). Doublets and multiplets were filtered out, as well as UMIs which had 2 or more hashtags associated with it, which resulted in 11, 682 remaining single cell transcriptomes. Additional QC included removing UMIs with excess mitochondrial reads (>5%), number of features less than 200 (low read counts) and more than 4200 (outliers). Finally, the standard Seurat pipeline running Normalize Data( ), Find Variable Features (selection method=“vst”, n features=2000), and Scale Data( ) was used. Score Jack Straw ( ) and Jack straw Plot was used to determine the number of dimensions before running Run PCA( ), Find Neighbours (dims=1:35), and Find Clusters(resolution=0.8). Run UMAP (dim=1:35, n neighbours=30) was used to first visualize the data, which revealed 13 clusters, of which two small clusters, cluster 11 and 12 were identified to be contaminating B cells and platelets. This was subsequently removed from the Seurat object, which now contains 11 distinct clusters, containing 11,347 cells. Mapping of a maturation score derived from Evrard & Kwok et al. 2018 (Evrard, M., et al., Development analysis of bone marrow neutrophils reveals populations specialised in expansion, trafficking, and effector functions, Immunity, 48, 364-379, 2018, the content of which is incorporated herein in its entirety) and Xie et al. 2020 (Xie X., et al., Single-cell transcriptome profiling reveals neutrophil heterogeneity in homeostasis and infection, Nature Immunology, Vol. 21, September 2020, pages 119-1133, the content of which is incorporated herein in its entirety) using FindModuleScore( ) was used in tandem with tissue identity to identify clusters corresponding to each maturation stage (Precursor, Immature and Mature), or belonging exclusively to the tumour. Similarly, a PMN-MDSC signature of 796 genes was obtained from our bulk RNAseq data was calculated for our Seurat dataset. Spliced and unspliced count matrices were obtained using Velocyto and exported as loom files for subsequent input into scVelo in Python 3.7. Velocity analysis was performed with scVelo using the default stochastic model and velocity vectors were projected into the UMAP embedding.

### Results

### Tumour Immature and Mature Neutrophil Subsets are Transcriptionally Distinct from their Counterparts in Control and Tumour-Bearing Mice

PMN-MDSCs have been classically defined as Gr-1^{hi}CD11b^{hi}Ly6G⁺ cells in tumour-bearing mice. However. these markers are also utilized in control mice to define neutrophils. Thus, it is unclear if all Gr-1^{hi}CD11b^{hi}Ly6G⁺ neutrophil populations identified in tumour-bearing mice across multiple organs including the blood, spleen and bone marrow should be classified as PMN-MDSCs alongside with populations infiltrating the tumour. In order to address this question, the inventors of the present disclosure performed RNA-seq of sorted immature and mature neutrophil populations from the blood, bone-marrow, spleen and tumour derived from our orthotopic pancreatic cancer model, as well as the equivalent populations from blood and bone marrow of control mice (non-tumour bearing). Utilizing principal component analysis (PCA) across the top 10% of genes with the highest variance in our RNA-seq, the inventors of the present disclosure show that tumour immature and mature neutrophil populations distinctly cluster away from all other neutrophil subsets (FIG. 1A). Spearman correlation analysis across all populations confirmed that tumour immature and mature neutrophil subsets had more closely correlated transcriptomes compared to the rest of the other subsets (FIG. 1B). Lastly, the inventors of the present disclosure identified 796 differentially expressed genes that were upregulated in tumour immature and mature neutrophils compared to the other neutrophil subsets (FIG. 1C), including genes previously attributed to PMN-MDSC function such as Arg1 and Nos2. Gene ontology and REVIGO analysis identified that these 796 genes were enriched for pathways governing apoptosis, chemotaxis, and secretion of pro-inflammatory and pro-angiogenic cytokines (FIG. 1D), which play important roles in PMN-MDSC survival, migration, and pro-tumorigenic function. This indicates that tumour neutrophils, but not neutrophils from the blood, spleen or bone marrow, share a functional transcriptome with PMN-MDSCs.

### Screening of Cell Surface Markers Identify dcTRAILR1 as a Unique Marker of a Subset of Tumour Mature Neutrophils Co-Expressing Immunosuppressive Markers

In order to find a theragnostic surface marker that could distinguish the PMN-MDSC population from the other neutrophil subsets in the tumour and other tissues, the inventors of the present disclosure evaluated the expression of 255 surface markers utilizing the LEGENDSCREEN™ kit (Biolegend) alongside 12 different backbone surface markers by flow cytometry in CD45⁺ cells from the tumour. The inventors of the present disclosure then utilized the InfinityFlow pipeline to predict co-expression of each tested surface marker and performed Uniform Manifold Approximation and Projection (UMAP) analysis to discriminate each of the different cell lineages. UMAP analysis revealed that neutrophils comprised up to 50% of all CD45⁺ cells within the tumour and were clustered into two distinct clusters that were not due to differential expression of CD101. Given that tumour neutrophils did not cluster into mature CD101⁺ and CD101⁻ immature subsets, the inventors of the present disclosure evaluated the dataset for markers that could clearly distinguish each cluster and stained other cell types minimally. The inventors identified dcTRAILR1, which stained one cluster exclusively within the neutrophil clusters (FIG. 2A).

The inventors of the present disclosure further validated dcTRAILR1 staining and saw the highest proportion of dcTRAILR1⁺ cells in Ly6G^{hi} neutrophils compared to immature neutrophils, monocytic Ly6C^{hi} MDSC and MHCII⁺F4/80⁺ tumour-associated macrophages (FIG. 2B). Furthermore, dcTRAILR1 was highly expressed in tumour mature neutrophils and not in other neutrophil subsets originating from the blood, spleen and bone marrow (FIG. 2C). The inventors of the present disclosure next further assessed co-expressed markers in the UMAP analysis and showed that dcTRAILR1 was co-expressed with surface markers known to be related to active immunosuppression of effector T cells (PdI-1, VISTA, Lilrb4), inhibitory receptors that negatively regulate innate inflammatory function (Dcir2, Pir-a/b, Clec12a), as well as the metabolic ectoenzymes CD39 and CD73 that create an immunosuppressive TME (FIG. 2D). In all, the inventors of the present disclosure illustrate that two populations of tumour-infiltrating neutrophils are present, and that dcTRAILR1 can serve as a specific cell surface marker to identify the putative, immunosuppressive PMN-MDSC within the tumour.

### dcTRAILR1 Expression Identifies the PMN-MDSC Population in Tumour Infiltrating Neutrophils

The findings thus far indicate that there is greater heterogeneity in tumour infiltrating neutrophil populations than previously proposed, given that the PMN-MDSC transcriptional signature is contained in both immature and mature tumour neutrophils, and dcTRAILR1 surface marker expression can identify a subset of tumour neutrophils expressing immunosuppressive markers. As such, to further dissect the heterogeneity within tumour infiltrating neutrophils, the inventors of the present disclosure carried out single-cell RNA sequencing (scRNAseq) on neutrophil populations from the bone marrow, blood, spleen and tumour of tumour-bearing mice (FIG. 3A). UMAP visualization (FIG. 3B) showed a clear separation of tumour neutrophils away from other neutrophils derived from the bone marrow, spleen and blood, which formed clusters based on their maturation stage (precursor, immature or mature). RNA Velocity analysis indicated that the differentiation trajectory in tumour neutrophils initiated in two vertexes of the tumour cluster and ended in a distinct tumour exit state (FIG. 3C). In contrast, in the other neutrophil subsets, differentiation followed an expected maturation trajectory which exited into a mature neutrophil phenotype (FIG. 3C).

In order to characterize the final differentiated state in tumour neutrophils, the inventors of the present disclosure mapped the PMN-MDSC enriched signature previously identified in the bulk RNAseq analyses (FIG. 1C) to the entire scRNAseq dataset. This PMN-MDSC signature score was enriched most highly in cells located in the tumour exit state (FIG. 3D), indicating that neutrophils that enter the tumour eventually acquire a new transcriptome to converge onto the PMN-MDSC phenotype. Lastly, the inventors of the present disclosure assess if dcTRAILR1 expression could identify the final PMN-MDSC population exemplified by the exit state. The inventors of the present disclosure found neutrophils in the exit state strongly expressed Tnfrsf23, the gene encoding dcTRAILR1 (FIG. 3E), and the highest Tnfrsf23 expression colocalized in cells showing the greatest enrichment for the PMN-MDSC signature score. Taken together, the results as described herein show that dcTRAILR1 expression can be a specific biomarker to identify the tumour-infiltrating neutrophils that have differentiated into PMN-MDSCs.

### Expression of the Human Decoy Receptor Ortholog, TRAIL-R3, in a Subset of Human Solid Tumours Correlates with Poor Patient Survival

Both human and mouse have two cell surface receptors that act as decoy receptors for TRAIL signalling due to the absence of functional downstream death-domain signalling. As such, they are suggested to have anti-apoptotic effects on the expressor cell. Conservation of amino acid sequences are minimal across human and mouse orthologs, but structurally and functionally, mouse dcTRAILR1 is similar to human TRAIL-R3 due to the presence of a GPI anchor and the absence of intracellular death domains (FIG. 4).

In order to assess if the TRAIL pathway could play a role in tumour progression, the inventors of the present disclosure assessed the expression of TRAIL and TRAIL-R3 in the PRECOG (Prediction of Clinical Outcomes from Genomic data) dataset generated at Stanford (Gentles et al., 2015). Across 25 solid tumour expression datasets collated in the PRECOG dataset, increased TRAIL expression within the tumour correlated strongly with favourable overall survival outcomes in 18 different cancers, including pancreatic ductal adenocarcinoma (PDAC) (FIG. 5A). We next assessed TRAIL-R3 expression and showed that 11 cancer types were correlated with adverse survival outcomes, with the highest z-score belonging to PDAC (FIG. 5B). Given that PMN-MDSCs are particularly enriched within PDAC, we anticipate that TRAIL-R3 expression will correlate strongly with the PMN-MDSC population.

To assess this, the inventors of the present disclosure evaluated a published scRNAseq dataset (Zilionis et al. 2019) of CD45+ immune populations in non-small cell lung cancer (NSCLC) (FIG. 6A). Utilizing the expression of CD10, the inventors of the present disclosure were able to distinguish mature and immature neutrophils within NSCLC tumour (FIG. 6B). The inventors of the present disclosure saw that similarly to the observations in the mouse PDAC models as described herein, the TRAIL-R3 decoy receptor was found within the mature neutrophil population (FIG. 6C). The inventors of the present disclosure further corroborated that the TRAIL-R3+ mature neutrophil population had co-expression of cell surface markers such as VISTA (FIG. 6D, in mouse—FIG. 2B), as well as genes such as ARG1 (FIG. 6E) and PTGS2 (FIG. 6F) annotated in the RNAseq (FIG. 1C). Overall, the inventors of the present disclosure identify the TRAIL-R3 decoy receptor as a PMN-MDSC marker in both mouse and human cancers. The present disclosure provides method for utilizing TRAIL-R3 as a PMN-MDSC marker, as well as a druggable target to deplete PMN-MDSCs in cancer.

The inventors of the present disclosure utilize an orthotopic pancreatic cancer preclinical mouse model, in which a tumour cell line containing two inactivating mutations against Kras and Tp53 (found in 80% and 70% of all human PDACs respectively) is injected directly into the mouse pancreas. Tumour growth and burden, as well as clinical presentation in this mouse model thus recapitulates human PDAC. The inventors had previously shown that both immature Ly6G+CD101− and mature Ly6G+CD101+ neutrophils are able to infiltrate the tumour tissue. Here, using transcriptomic analysis, the inventors of the present disclosure showed that tumour infiltrating immature and mature neutrophil subsets share a distinct transcriptional signature which distinguished them from their counterparts in the circulation (blood), spleen and bone marrow of tumour bearing and control mice. The tumour transcriptional signature was enriched for genes involved in PMN-MDSC immunosuppressive function as well as genes enhancing tumour progression, indicating that PMN-MDSCs are contained within the tumour infiltrating population. Screening of surface markers revealed that tumour mature neutrophils can be separated by the expression of dcTRAILR1, and that dcTRAILR1+ neutrophils had co-expression of immunosuppressive and inhibitory markers. Deeper analysis by scRNAseq further validates the use of dcTRAILR1 to identify the putative PMN-MDSCs amongst a heterogenous tumour infiltration neutrophil population. The inventors of the present disclosure found that dcTRAILR1+ neutrophils represent the putative PMN-MDSC population, and this identification strategy would be useful for functional studies in pre-clinical models. Extension of studies to the human ortholog, TRAIL-R3, in human PMN-MDSCs will assess the suitability of TRAIL-R3 as a depleting marker for PMN-MDSCs in human cancer singly or in combination with other immunotherapy approaches.

### APPLICATIONS

Embodiments of the methods disclosed herein provide a fast, efficient and cheap way of identifying, sorting, characterising immunosuppressive neutrophils.

Advantageously, the methods as described herein can be used to identify PMN-MDSCs in mouse preclinical cancer models. As described herein, the identification of anti-apoptotic markers such as dcTRAILR1 and/or TRAIL-R3 in neutrophils advantageously sort immunosuppressive neutrophils from effector neutrophils.

Even more advantageously, as dcTRAILR1 is specific only for PMN-MDSCs and not for other circulating, spleen or bone marrow neutrophils, the anti-apoptotic marker allows for the selective ablation of PMN-MDSCs, which may facilitate the sequelae of therapy-induced neutropenia.

Even yet advantageously, as dcTRAILR1 and/or TRAIL-R3 is a cell surface marker, they can be targeted by antibodies that is optimized for neutralizing or depleting activity.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of detecting and/or characterising an immunosuppressive neutrophil comprising determining and/or measuring the expression of one or more anti-apoptotic marker in a neutrophil population.

2. The method of claim 1, wherein the immunosuppressive neutrophil is a polymorphonuclear myeloid-derived suppressor cell (PMN-MDSC).

3. The method of claim 1, wherein the anti-apoptotic marker is TNF-related apoptosis-inducing ligand (TRAIL)-receptor 3 (TRAIL-R3/TNFRSF10C; in human) and/or decoy TNF-related apoptosis-inducing ligand (TRAIL)-receptor (dcTRAILR1; in mouse).

4. The method of claim 1, wherein the method further comprises: determining and/or measuring the expression of one or more markers related and/or capable of inducing immunosuppression of an immune cell.

5. The method of claim 1, wherein the markers related and/or capable of inducing immunosuppression of effector immune cells comprises one or more of the following markers: CD274 (PD-L1), VSIR (VISTA), LILRB4, ARG1, PTGS2 (COX2), or NOS2.

6. The method of claim 1, wherein the method further comprises: determining and/or measuring the expression of one or more markers that negatively regulate inflammatory function, optionally negatively regulate innate inflammatory function.

7. The method of claim 1, wherein the markers that negatively regulate innate inflammatory function comprises one or more of the following markers: Dcir2, Pir-a/b, or Clec12a.

8. The method of claim 1, wherein the method further comprises: determining and/or measuring the expression of one or more markers of metabolic ectoenzymes that create immunosuppressive tumour microenvironment (TME).

9. The method of claim 1, wherein the \markers of metabolic ectoenzymes that create the immunosuppressive TME comprises one or more of the following markers: CD39, or CD73.

10. The method of claim 1, wherein the method further comprises: determining and/or measuring the expression of markers comprising one or more of the following markers:
(in mouse) CD11b+, Gr-1hi, Ly6Clow, Ly6G+, CD101+/−, optionally wherein CD101+ refers to mature neutrophil population and/or CD101− refers to immature neutrophil population and/or
(in humans) CD11b+, CD101+, CD66b+, CD15+, CD101+, CD16+/−, CD10+/−, optionally wherein CD16low/−CD10− refers to immature neutrophil population, and/or CD16+CD10+ refers to the mature neutrophil population, and/or
(in RNAseq transcriptomic profiles) MMP8, MMP9, S100A9, S100A8, CSF3R, CXCR2.

11. The method of claim 1, wherein the method further comprises determining and/or measuring the pro-angiogenic ability of the cell, optionally, the method determines and/or measures markers comprising one or more of the following markers: MMP9, IL-6, VEGFα production, and/or combination thereof.

12. The method of claim 1, wherein the method further comprising sorting a plurality of cells into subpopulations, the method comprising clustering the heterogenous population into subpopulations based on their expression of one or more of the following:
(in mouse) Lin−, MHCII−, F4/80, Ly6Cint, Ly6G+, dcTRAIL-R1+, ARG-1+, VISTA+, and/or
(in human) CD11b+, CD101+, CD66b+, CD15+, TRAIL-R3+, ARG-1+, PTGS2+, and the like.

13. A method of evaluating the progression of a proliferative disease in a subject, the method comprising:
determining/measuring an activity, an amount and/or a proportion of an immunosuppressive neutrophil in a first sample from the subject; and
comparing the activity, the amount and/or the proportion of an immunosuppressive neutrophil to those of a second sample from the subject, the second sample being obtained from subject at a later timepoint than the first sample,
wherein an increased activity, increased amount and/or increased proportion of an immunosuppressive neutrophil in the second sample as compared to the first sample is indicative of a worsening of the condition in the subject, and/or
wherein a reduced activity, reduced amount and/or reduced proportion of an immunosuppressive neutrophil in the second sample as compared to the first sample is indicative of an improvement of the condition in the subject.

14. The method of claim 13, further comprising evaluating the efficacy of a treatment regimen (e.g. efficacy of a drug) for a proliferative disease in a subject, the evaluating comprising:
comparing the activity, the amount and/or the proportion of an immunosuppressive neutrophil to those of a second sample from the subject, the second sample being obtained from subject at a later timepoint in the treatment regimen than the first sample,
wherein the treatment regimen is considered when the second sample has a reduced activity, reduced amount and/or reduced proportion of the immunosuppressive neutrophil as compared to the first sample and non-effective if otherwise.

15. The method of claim 13, further comprising treating the subject in need thereof, wherein the treating comprising:
administering to the subject a composition that is capable of decreasing an activity, an amount (or a level) and/or a proportion of an immunosuppressive neutrophil.

16. The method of claim 1, wherein the sample is a tissue biopsy, such as a tumour and/or cancer biopsy.

17. A kit for characterising an immunosuppressive neutrophil comprising one or more reagent that determines and/or measures the expression of one or more anti-apoptotic receptor markers comprising TRAIL-R3 and/or dcTRAILR1.

18. The method of claim 13, wherein the sample is a tissue biopsy, such as a tumour and/or cancer biopsy.
